(19) United States
(12) Patent Application Publication
(10) Pub. No.: US 2009/0326558 A1
(43) Pub. Date: Dec. 31, 2009

(54) CLIPPING DEVICE

(76) Inventors: Shengfu Cui, Kanagawa (JP); Takayuki Iida, Kanagawa (JP)

Correspondence Address: BIRCH STEWART KOLASCH & BIRCH, PO BOX 747, FALLS CHURCH, VA, 22040-0747, US

(21) Appl. No.: 12/493,975

(22) Filed: Jun. 29, 2009

(30) Foreign Application Priority Data
Jun. 30, 2008 (JP) 2008-170876
Sep. 29, 2008 (JP) 2008-251205

(51) Int. Cl. A61B 17/10

(52) U.S. Cl. 606/143; 606/142

(57) ABSTRACT

A clipping device comprises: a clip having a pair of arm portions diverging elastically; and a tubular clamping member fitted onto the pair of arm portions of the clip, each of the pair of arm portions including a projection portion which is formed in a vicinity of a forward end portion thereof to have a width inhibiting passage of the clamping member, and an engagement portion which is formed in a region adjacent to a proximal end side of the projection portion to be engaged with the clamping member, the clamping member being engaged with the engagement portion of each of the pair of arm portions to maintain clamping of the clip so that the pair of arm portions holds an object to be treated.

### BACKGROUND OF THE INVENTION

The present invention relates to an endoscopic clipping device used for stopping bleeding, closing a puncture, etc. in a living body or the like.

An endoscopic clipping device causes a clip to protrude from the forward end of an endocope inserted into a living body to pinch a bleeding portion or a portion to be treated after the removal of the lesion tissue with the clip, thereby stopping the bleeding or closing the puncture. Generally, as such a medical clip, there is known a clip having a structure in which removal is not easily performed once the clip has been clamped.

For example, in JP 05-505732 A, there is described a suture device having a plurality of legs, which includes two elongated tissue engaging and penetrating legs opposed to each other, and a locking/tying member partially covering the two legs and moving along the legs to a distal end side (forward end side) of the legs so as to bring the two legs close to each other to thereby join the same together, and in which a plurality of inclined teeth or spines are provided on outer surfaces of the two legs holding the living body, and the locking/tying member is provided with an internal engaging means such as inclined teeth, whereby the locking/tying member can be selectively moved toward distal ends (forward ends) of the legs. In the suture device, the legs and the engaging means of the locking/tying member interact with each other, whereby the locking/tying member which has moved to the distal end side once is not easily detached.

Meanwhile, after the elapse of a fixed period of time from clipping, it is necessary in some cases to remove the clip which has achieved a purpose to stop bleeding. Further, during treatment such as stopping bleeding by the clip, it is necessary in some cases to correct a position of a portion held by the clip once. In this context, in JP 2007-125264 A, there is described a clip removing device for removing a clip which has held a living tissue once.

The clip removing device disclosed in JP 2007-125264 A includes a plurality of clamping ring retaining arms for performing an opening/closing movement so as to retain a clamping ring, and a clip pushing-out member for pushing out the clip forward relatively to the clamping ring which is retained by the clamping ring retaining arms. The clip is made of a material having elasticity, and formed into a beak-like shape opening forward. In a state in which the clamping ring is retained by the clamping ring retaining arms, the clip is pushed out by the clip pushing-out member, the clip being closed by sliding forward the tubular clamping ring covering the clip from the rear thereof so as to be placed in a state of biting the living tissue. As a result, the clip is opened due to its own elasticity, and removed from the living tissue.

In the suture device of JP 05-505732 A, the locking/tying member can be inhibited from moving to the rear of the legs by the engaging means such as the inclined teeth provided between the legs and the locking/tying member, and hence it is possible to maintain a suture state at the forward ends of the legs. However, the suture device sutures the living tissue by bringing the forward ends of the legs into contact with each other, but does not pinch and hold the living tissue between the forward ends thereof. Thus, it is not determined which position of the legs the locking/tying member moves to, and a predetermined fit-engagement force (holding force) is not exerted at the forward ends of the legs. Further, in the suture device, the legs which have held the living tissue once are not designed to be removed from the living tissue.

Meanwhile, in the clip according to the clip removing device of JP 2007-125264 A, the clip and the clamping ring do not include the engaging means such as protrusions, and the clip is clamped solely by pulling the clip into the clamping ring. Therefore, contrary to during clamping, only by applying a force for pushing out the clip with respect to the clamping ring, it is possible to cancel clamping of the clip by the clamping ring. However, in order to obtain still larger clamping force or precise and reliable holding force, as in the case of JP 05-505732 A, when the engaging means such as the inclined teeth is provided between the clip and the clamping ring, the clamping ring can not be moved to the rear of the clip in the state of clamping the clip. Further, with the clip removing device disclosed in JP 2007-125264 A, the clip which has held the living tissue once can not be removed.

### SUMMARY OF THE INVENTION

The present invention has been made for solving the problems of conventional technologies, and therefore has an object to provide a clipping device capable of reliably maintaining a state achieved after a designed fit-engagement force is easily and precisely exerted on claw portions, and capable of canceling holding by the claw portions when a clip which has held a living body once needs to be removed.

A clipping device according to a first aspect of the present invention comprises: a clip having a pair of arm portions diverging elastically; and a tubular clamping member fitted onto the pair of arm portions of the clip, for releasing the clip when being situated on a proximal end side of the pair of arm portions so as to cause the pair of arm portions to diverge, and for clamping the clip when being situated on a forward end side of the pair of arm portions so as to cause the pair of arm portions to close, each of the pair of arm portions including a projection portion which is formed in a vicinity of a forward end portion thereof to have a width inhibiting passage of the clamping member, and an engagement portion which is formed in a region adjacent to a proximal end side of the projection portion to be engaged with the clamping member, the clamping member being engaged with the engagement portion of each of the pair of arm portions to maintain clamping of the clip so that the pair of arm portions holds an object to be treated.

A clipping device according to a second aspect of the present invention comprises: a sheath having a proximal end portion and a forward end portion; a plurality of clips which are loaded into the forward end portion of the sheath while being engaged with other clips connected together in front and back directions, and each of which comprises a pair of arm portions diverging elastically; a plurality of tubular clamping members corresponding to the plurality of clips and fitted into the sheath so as to be capable of advancing and retreating, each being fitted onto the pair of arm portions of the corresponding clip, each releasing the corresponding clip when being situated on a proximal end side of the pair of arm portions so as to cause the pair of arm portions to diverge, and each clamping the corresponding clip when being situated on a forward end side of the pair of arm portions so as to cause the pair of arm portions to close; a plurality of retaining members corresponding to the plurality of clamping members and connected to the corresponding clamping members, and each covering an engagement portion of the clip, onto which the corresponding clamping member is fitted, so as to maintain the plurality of clips in a connected state; and a manipulating wire connected to a rearmost one of the plurality of clips, for pulling a clip string constituted by the plurality of clips, each of the pair of arm portions of each clip including a projection portion which is formed in a vicinity of a forward end portion thereof to have a width inhibiting passage of the clamping member, and an engagement portion which is formed in a region adjacent to a proximal end side of the projection portion to be engaged with the clamping member, each of the clamping members being engaged with the engagement portion of each of the pair of arm portions of the corresponding clip to maintain clamping of the clip.

### BRIEF DESCRIPTION OF THE DRAWINGS

FIGS. 1A and 1B are partial cross-sectional views of a successive clipping device according to a first embodiment of the present invention;

FIG. 2 is a perspective view of a clip;

FIG. 3 is a view illustrating a relation between a forward end opening of a connection ring and claw portions;

FIGS. 4A to 4C are a front view, a cross-sectional view, and a bottom view of the connection ring, respectively;

FIG. 5A is a perspective view illustrating the clip and the connection ring immediately before completion of clamping;

FIG. 5B is an enlarged partial cross-sectional view illustrating projection portions and micro projections upon completion of clamping;

FIG. 6 is an enlarged cross-sectional view of one of the micro projections;

FIGS. 7A and 7B are partial cross-sectional views illustrating states of the clips and the connection ring during clipping operation of the first embodiment in stepwise, respectively;

FIGS. 8A to 8C are schematic views illustrating states of the clip after completion of clamping by a clamping portion of the connection ring, respectively;

FIGS. 9A to 9C are graphs showing a relation between a sliding amount of the connection ring and a fit-engagement force generated between the claw portions of the clip when the clamping state illustrated in each of FIGS. 8A to 8C is obtained;

FIGS. 10A and 10B are an entire perspective view of a clip used in a second embodiment and an enlarged partial perspective view thereof, respectively;

FIG. 11 is a cross-sectional view of a connection ring used in the second embodiment;

FIGS. 12A and 12B are partial cross-sectional views illustrating states of the clip and the connection ring during clipping operation of the second embodiment in stepwise, respectively;

FIG. 13A is a view illustrating a positional relation between a clamping portion of the connection ring and arm portions of the clip at the time of pulling a manipulating wire in the second embodiment;

FIG. 13B is a cross-sectional view taken along the dashed arrow of FIG. 13A;

FIG. 14A is a cross-sectional view illustrating a positional relation between the clamping portion of the connection ring and the arm portions of the clip upon completion of clamping in the second embodiment; and

FIG. 14B is a cross-sectional view taken along the dashed arrow of FIG. 14A.

### DETAILED DESCRIPTION OF THE PREFERRED EMBODIMENTS

The present invention is applicable to any of a single clipping device using one clip and a successive clipping device connecting a plurality of clips to successively use the clips. Herein, embodiments in which the successive clipping device is applied are described.

### First Embodiment

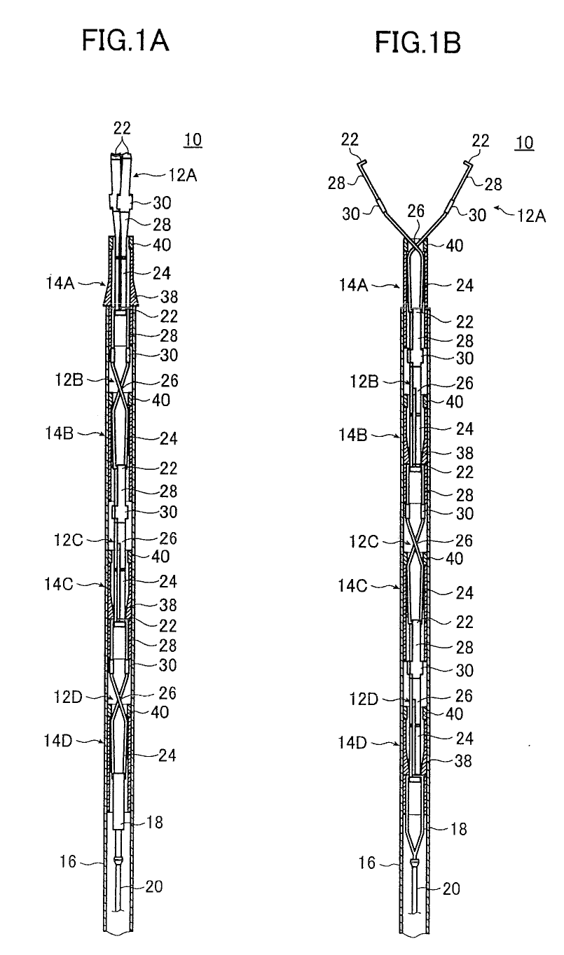

FIGS. 1A and 1B illustrate a successive clipping device 10 according to a first embodiment of the present invention. FIG. 1B is a view as seen from an angle differing from FIG. 1A by 90 degrees.

The clipping device 10 is a successive clipping device capable of successively using clips, and includes a plurality of clips 12 (12A, 12B, 12C, and 12D), a dummy clip 18 connected to the rearmost clip 12D, a manipulating wire 20 connected to the dummy clip 18, connection rings 14 (14A, 14B, 14C, and 14D) that cover engagement portions of two clips 12 continuous with each other in front and back directions to maintain the connection state of the clips 12. These components of the clipping device 10 are fitted in a sheath 16. FIGS. 1A and 1B illustrate an initial state immediately before the start of clipping operation by the foremost clip 12A.

One clip 12 and one connection ring 14 corresponding to the clip 12 form one endoscopic bleeding stop clip member, and the clipping device 10 includes a plurality of such bleeding stop clip members loaded into the interior of the distal end portion of the elongated sheath 16. The terminal end of the successive bleeding stop clip members is engaged with the dummy clip 18, and the manipulating wire 20 connected to the dummy clip 18 extends to the rear end portion of the sheath 16 to be connected to a manipulating portion (not shown). The manipulating wire 20 is pulled from the manipulating portion by a predetermined length to move the dummy clip 18 in one direction by the predetermined length, whereby a series of the plurality of the clips 12 are moved by the same amount with respect to the connection ring 14 situated at the forward end of the sheath 16. As a result, clipping for stopping bleeding, marking, etc. is effected by the foremost clip 12. After the clipping by the foremost clip 12 has been completed, the sheath 16 is pulled to the manipulating portion side by the predetermined length or the manipulating wire 20 is pushed thereto, whereby the next clip 12 is placed in a usable state (standby state), thus making it possible to effect clipping successively.

While in FIGS. 1A and 1B the foremost clip 12A protrudes from the forward end of the sheath 16, when loading the clips 12 into the sheath 16, setting is effected such that the foremost clip 12A is completely accommodated within the sheath 16. Further, while in the first embodiment the 4-successive clipping device in which 4 clips 12 are loaded, the number of clips 12 may be any number not less than two.

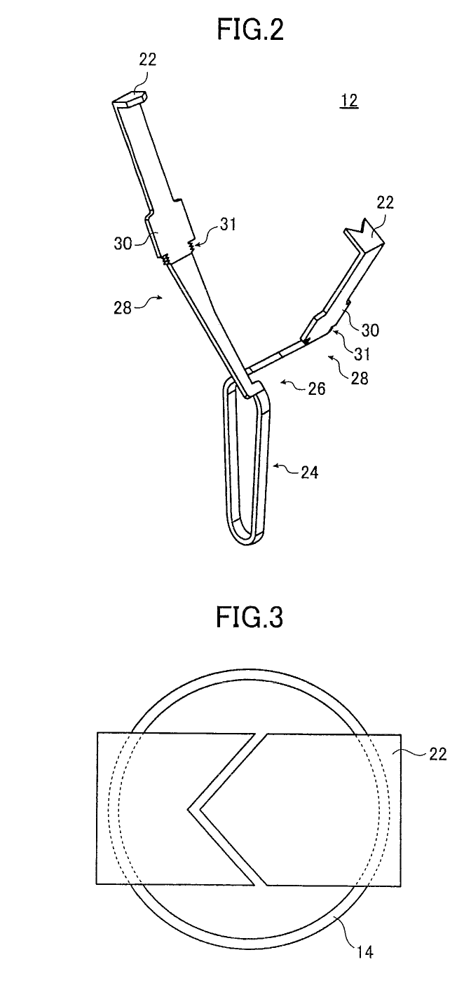

FIG. 2 is a perspective view of the clip 12. The clip 12 is a closed clip having a turned portion 24 turned by 180 degrees with respect to claw portions 22. That is, in forming the clip 12, a single elongated plate is bent by 180 degrees to form a closed end, and then both ends thereof are caused to cross each other. Further, the end portions are bent so as to be opposed to each other, thereby forming the claw portions 22, 22 to two open ends. On the open-end side of the crossing portion 26, there exist arm portions 28, 28, and, on the closed-end portion thereof, there exists the turned portion 24. At the central portion of each arm portion 28, 28, there are formed a partially widened projection portions 30, 30. Further, micro projections 31, 31 are formed at edge portions on both sides of each of the projection portions 30, 30 of the lower side of the figure. The clip 12 may be formed of a metal with biocompatibility. For example, it is possible to use SUS 631, which is a spring stainless steel.

In the clip 12, the forward end portion (clamping portion 40 described later) of the connection ring 14 fitted onto the crossing portion 26 moves by a predetermined amount toward the claw portions 22, 22 while pressurizing the arm portions 28, 28, whereby the arm portions 28, 28 and the claw portions 22, 22 are closed and moved to a portion abutted by the projection portions 30, with the claw portions 22, 22 exerting a predetermined fit-engagement force.

To reliably pinch an object of a bleeding portion, the claw portions 22, 22 are preferably formed as V-shaped male type and female type ones. Further, as illustrated in FIG. 2, the arm portions 28 of the clips 12 gradually increase in width from the crossing portion 26 toward the projection portions 30.

FIG. 3 is a view as seen from the forward end side of the clip 12, illustrating a relation in size between the forward end opening of the connection ring 14 and a pair of claw portions 22, 22 when the claw portions 22, 22 of the clip 12 are closed by the connection ring 14. As illustrated in FIG. 3, the total length (length in facing direction) of both the claw portions 22, 22 when the pair of claw portions 22, 22 of the clip 12 is closed is larger than the maximum inner diameter of the opening of the forward end of the connection ring 14 (clamping portion 40 described below). Therefore, the connection ring 14 is moved toward the claw portions 22, 22 to progress clamping of the clip 12, whereby the arm portions 28, 28 of the clip 12 are pressed against the inner wall of the connection ring 14 due to bending rigidity (elastic force) of the clip 12.

Specific examples of dimensions of the connection ring 14 and the clip 12 include the total length 1.4 mm in the facing direction of the pair of claw portions 22, 22, the width 0.7 mm of the claw portion 22, and the maximum inner diameter 1.2 mm of the forward end portion of the connection ring 14.

The projection portions 30 have a width larger than that of the portions of the distal end side openings and the proximal end side openings of the connection ring 14 abutted by the projection portions 30. Thus, while the portions of the clip 12 other than the projection portions 30 can enter the interior of the connection ring 14, the projection portions 30 cannot enter the interior either from the distal end side or the proximal end side of the connection ring 14.

The projecting portions (hereinafter referred to as lower ends), which are provided on the proximal end side of the projection portions 30 and have a width not allowing to enter the connection ring 14, function to determine the upper limit point in the moving range of the connection ring 14. The projecting portions are provided so as to be matched with the moving position of the forward end of the connection ring 14 in which a fit-engagement force previously set in the claw portions 22, 22 of the clip 12 is obtained by the movement of the connection ring 14 when the clip 12 is clamped by the connection ring 14.

The forward end (clamping portion 40) of the connection ring 14 is moved to the position of being brought into contact with the projection portions 30 of the clip 12, whereby the clip 12 can exert a predetermined fit-engagement force, for example, the design upper limit value of the fit-engagement force in the claw portions 22, 22.

Further, by the provision of the projection portions 30 to the clip 12, the connection ring 14 is prevented from moving to the forward end side by the predetermined amount or more, and it is possible to avoid a problem that the clip 12 is excessively clamped, or a problem that deformation of the clip 12 occurs to disable to appropriately hold a living tissue.

Micro projections 31 are provided to edge portions on both sides of each of the arm portions 28 in a predetermined region on the proximal end side (rear end side) of each of the projection portions 30. By a frictional force with the metal forward end portion (clamping portion 40) of the connection ring 14 moved to the position of being brought into contact with the projection portions 30, and in addition, by friction (galling) of metal portions, the micro projections 31 retain the connection ring 14 at the above-mentioned position. The preferred embodiment and operation of the micro projections 31 are described later in detail.

For example, when punching a strap-shaped plate used as a material of the clip 12, portions corresponding to the projection portions 30 and the micro projections 31 are previously provided, and the punched plate is bent as described above and the micro projections 31 are processed into a desired shape, whereby the projection portions 30 and the micro projections 31 can be formed. Note that, the thickness of the portions corresponding to the projection portions 30 may be different from that of the arm portions 28, or portions folded inward with respect to a diverging direction of the clip 12 may be provided on the end portions thereof in the width direction (horizontal direction). In this case, the strength of the projection portions 30 can be increased.

As illustrated in FIGS. 1A and 1B, the claw portions 22 of the second clip 12B are engaged with the turned portion 24 of the first clip 12A and retained by the connection ring 14A in a closed state, whereby the first clip 12A and the second clip 12B are connected together. As illustrated in FIG. 1A, the claw portions 22, 22 of the second clip 12B are engaged with the turned portion 24 of the first clip 12A in a direction orthogonal thereto, with the first clip 12A and the second clip 12B being differing in orientation by 90 degrees. Similarly, the clips 12C and 12D are connected together, with their orientations alternately differing by 90 degrees.

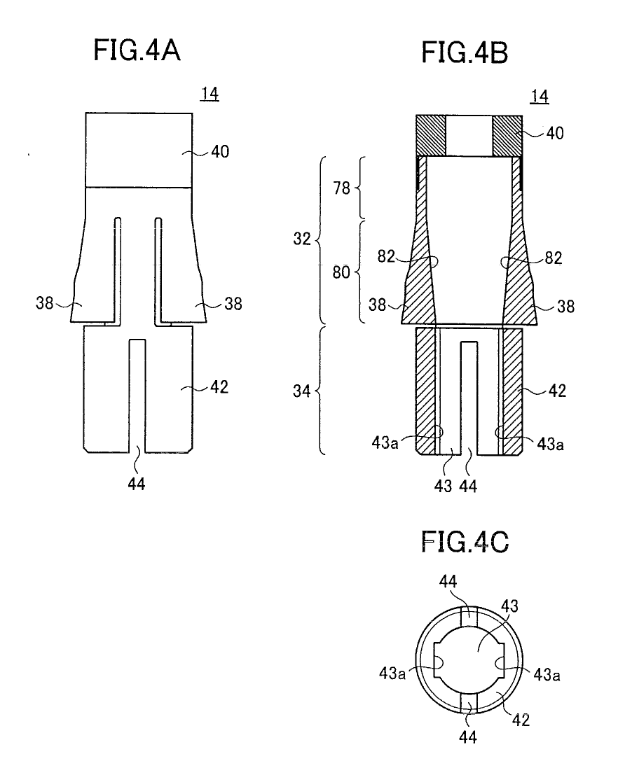

Each connection ring 14 is fitted into the sheath 16 so as to be capable of advancing and retreating while covering the engagement portion between the front and rear two clips 12 and maintaining their connected state. That is, the outer diameter of the connection rings 14 is substantially the same as the inner diameter of the sheath 16 so that they can smoothly advance and retreat within the sheath 16 as the clips 12 move. FIGS. 4A through 4C illustrate the schematic construction of each connection ring 14.

The connection ring 14 includes a clamping portion 40 and a retaining portion 42. In the connection ring 14, the clamping portion 40 formed of metal is fixed to the forward end of the retaining portion 42 formed of resin, and the two members form an integral structure. The retaining portion 42 formed of resin serves to maintain the connected state and to retain the clip within the connection ring, and the clamping portion 40 formed of metal serves to clamp the clip.

The clamping portion 40 is a cylindrical (ring-like) metal component mounted to the forward end side of the connection ring 14, and has a hole whose inner diameter is larger than the width of the portion of the clip 12 in the vicinity of the crossing portion 26 and smaller than the width of the projection portions 30. Thus, while the clamping portion 40 can move in the vicinity of the crossing portion 26 of the clip 12 it retains, it cannot be detached to the forward end side beyond the projection portions 30. That is, the projection portions 30 function as a stopper determining the movement limit of the connection ring 14 advancing with respect to the clip 12.

The clamping portion 40 moves from the crossing portion 26 toward the projection portions 30, with the arm portions 28 of the clip 12 increasing in width, whereby it closes the arm portions 28, 28 of the diverging clip 12 to effect fixation and clamping. As the material of the clamping portion 40, a metal with biocompatibility, for example, a stainless steel SUS 304 is used. By forming the clamping portion 40 of metal, it is possible to exert a frictional force, which serves as the clamping force of the metal clip 12 between the clamping portion 40 and the micro projections 31 of the metal clip 12.

The retaining portion 42 is a schematically cylindrical (ring-like) component formed by resin molding. The retaining portion 42 has a first region 32 retaining the preceding clip 12 and a second region 34 which is a connection retaining region retaining the next clip 12 while connected to the preceding clip.

The first region 32 has a large circular hole capable of accommodating the turned portion 24 of the preceding clip 12 and larger than the hole of the clamping portion 40. On the outer surface of the forward end portion of the first region 32, there is formed a stepped portion onto which the clamping portion 40 is to be fitted, and the clamping portion 40 and the retaining portion 42 are fit-engaged with each other through close fit such that they are not detached from each other while loaded in the sheath 16 and during clipping manipulation. Further, the first region 32 has skirt portions 38 each diverging while inclined in a skirt-like fashion with respect to the axis of the connection ring 14 main body.

The forward end side, that is, the upper base portion of each skirt portion 38 as seen in FIGS. 4A and 4B is connected to the main body of the retaining portion 42, whereas the lower diverging portion thereof is partially separated from the main body to be radially diverged or closed. Two skirt portions 38 are formed so as to be separated from each other by 180 degrees at the same position in the pulling direction for the clip 12, that is, in the vertical direction in FIGS. 4A and 4B.

When in a state in which no external force is being imparted thereto, the skirt portions 38, 38 are diverged in a skirt-like fashion as illustrated in FIG. 4A. At this time, the interior of the first region 32 of the retaining portion 42 forms a columnar space as illustrated in FIG. 4B. When loading the connection rings 14 into the sheath 16, the following takes place: in the case, for example, of the second connection ring 14B illustrated in FIG. 1B, the skirt portions 38 are pushed in to enter the internal space, and the inner peripheral side portions of the skirt portions 38 pressurize the side surface (edge portion) of the turned portion 24 of the clip 12B retained by the first region 32, thus retaining the clip 12B such that it does not move in the rotating direction and the advancing/retreating direction within the connection ring 14B.

As in the case of the first connection ring 14A illustrated in FIG. 1A, the skirt portions 38, 38 extend beyond the forward end of the sheath 16 and are opened, releasing the retention of the clip 12A and becoming wider than the inner diameter of the sheath 16 to prevent the connection ring 14A from retreating into the sheath 16. In this state, the manipulating wire 20 is pulled, and the clip 12A retreats, whereby the connection ring 14A advances relative to the clip 12A to clamp the clip 12A.

Thus, it is necessary for the skirt portions 38 to have elasticity so that they can be closed inwardly within the sheath 16 and widen in a skirt-like fashion when they get out of the forward end of the sheath 16. At the same time, it is also necessary for the skirt portions 38 to exhibit rigidity enabling them to retain the clip 12 within the sheath 16 and to withstand the repulsive force of the clamping force of the clip 12 at the forward end of the sheath 16.

From the above viewpoints, as the material of the retaining portion 42, there is used a material exhibiting biocompatibility and providing the requisite elasticity and rigidity for the skirt portions 38. As for their configuration, it is determined so as to satisfy the requirements in terms of elasticity and rigidity for the skirt portions 38. As the material of the retaining portion 42, it is possible to use, for example, polyphenylsulfone (PPSU). From the viewpoint of ease of production, it is desirable for the retaining portion 42 to be formed as an integral molding.

The second region 34 is provided on the proximal end side of the first region 32. The succeeding clip 12 engaged with the clip 12 retained by the first region 32 is retained in a state in which the claw portions 22, 22 thereof are closed while holding the closed end (tail portion) of the turned portion 24 of the preceding clip 12 therebetween.

The length of the second region 34 is substantially equal to the movement length required for the clamping portion 40 set at the initial position with respect to the clip 12 to move until the clamping of the clip 12 is completed. That is, while the clip 12 retreats relative to the connection ring 14 to be clamped, the second region 34 of the connection ring 14 maintains the connection between the two clips 12, 12 retained therein, allowing the pulling force of the rear clip 12 to be transmitted to the front clip 12, and when the clamping has been completed, the engagement portion of the two clips 12, 12 is detached from the second region 34, thereby canceling the connection between the clips 12, 12.

As illustrated in FIG. 4C, the second region 34 has a hole 43 having the same inner diameter as that of the first region 32, and further, two grooves (recesses) 43a opposed to each other are formed. The grooves 43a, 43a can accommodate the arm portions 28, 28 of the clip 12 retained in the second region 34, with the claw portions 22, 22 being closed. Further, in the second region 34, slits 44 that are cut in from the proximal end are formed at two positions.

The grooves 43a, 43a are provided at two positions in the direction in which the claw portions 22 of the clip 12 retained in the second region 34 are opened and closed (horizontal direction in FIG. 4). The plate surfaces of the arm portions 28, 28 of the clip 12 retained in the second region 34 abut the inner walls of the grooves 43a, 43a. The width (opening width) of the grooves 43a is slightly larger than the maximum width of the arm portions 28 of the clip 12, and the distance from the wall surface of one groove 43a to the wall surface of the other groove 43a is substantially equal to the sum total of the lengths of the two claw portions 22, 22 of the clip 12 (length in the diverging direction). The width of the grooves 43a is smaller than the width of the projection portions 30 formed on the arm portions 28. Thus, the projection portions 30 of the clip 12 retained in the second region 34 cannot enter the grooves 43a.

Note that the distance between the wall surfaces of the two grooves 43a is such that the engagement between the turned portion 24 of the preceding clip 12 and the claw portions 22, 22 of the next clip 12 is not canceled, and the distance is smaller than the sum total of the lengths of the two claw portions 22, 22 and the width of the portion of the turned portion 24 engaged with the claw portions 22, 22.

The engagement portion between the two clips 12, 12 is retained in the portion of the second region 34 close to the boundary between the second region 34 and the first region 32. Inside the sheath 16, the turned portion 24 of the preceding clip 12 (e.g., the clip 12B in the connection ring 14B illustrated in FIG. 1B) is retained by the closed skirt portions 38 in the first region 32, and hence the advancing/retreating movement and rotating movement of the clip is restrained. The next clip 12 (e.g., the clip 12C in the connection ring 14C illustrated in FIG. 1B) engaged with the preceding clip 12 is retained in an orientation differing by 90 degrees from the preceding clip by the rectangle grooves 43a of the second region 34, whereby rotating movement of the clip is restrained, and the clip is engaged with the preceding clip restrained in advancing/retreating movement, thereby restraining the advancing/retreating movement thereof. That is, the engagement portion between the front and rear clips is retained by the connection ring 14 with very little play.

The slits 44 are formed at two positions deviated from the skirt portions 38, 38 by 90 degrees so as to be shallower than the upper end of the second region 34. In other words, the slits 44 are provided at positions deviated by 90 degrees from the direction in which the clips 12 retained by the second region 34 are diverged.

Due to the provision of the slits 44, the connection ring 14 is improved in terms of flexibility, and the clipping device 10 can pass a curved portion of small curvature. Further, due to the provision of the slits, the hem (proximal end portion) of the connection ring 14 is partially turned up, and hence, when the front and rear clips 12 are connected together prior to the loading of the clips 12 into the sheath 16, the connection is advantageously facilitated through the turning of the hem of the connection ring 14.

The slits 44 are preferably situated so as to be shallower than the skirt portions 38. Thus, a substantial reduction in the strength of the connection ring 14 can be prevented.

Further, the depths of the slits 44 are preferably shallower than the position of the rear end of the clip 12 retained in the first region 32. That is, shallower than the engagement position of the clips 12, 12, and hence, also in the connection clip unit prior to the loading into the sheath 16, it is possible to maintain the retention of the clip 12 in the second region 34 of the connection ring 14.

As illustrated in FIGS. 1A and 1B, the claw portions 22 of the second clip 12B are engaged with the turned portion 24 of the first clip 12A, and the engagement portion is retained by the connection ring 14A. The claw portions 22, 22 of the second clip 12B are retained in the closed state by the inner wall of the connection ring 14A (second region 34 thereof). As a result, the connection of the first clip 12A and the second clip 12B is maintained. Similarly, the connection of the second clip 12B and the third clip 12C is maintained by the connection ring 14B, the connection of the third clip 12C and the fourth clip 12D is maintained by the connection ring 14C, and the connection of the fourth clip 12D and the dummy clip 18 is maintained by the connection ring 14D.

The rearmost clip 12D is engaged with the dummy clip 18, which is not used for clipping. The dummy clip 18 has a resilient portion of a configuration similar to that of the open end side half as from the crossing portion 26 of the clip 12. The resilient portion is engaged with the turned portion 24 of the clip 12D, with the claw portions 22 thereof being closed, and releases the clip 12D when the claw portions 22 are opened. At the proximal end portion of the dummy clip 18, the manipulating wire 20 is stably connected.

The sheath 16 is formed, for example, of a flexible coil sheath formed through intimate winding of metal wire. The inner diameter of the sheath 16 is one allowing canceling of the engagement between the turned portion 24 of the preceding clip 12 and the claw portions 22, 22 of the next clip 12. That is, the inner diameter of the sheath 16 is larger than the sum total of the lengths of the two claw portions 22, 22 and the width of the portion of the turned portion 24 engaged with the claw portions 22, 22.

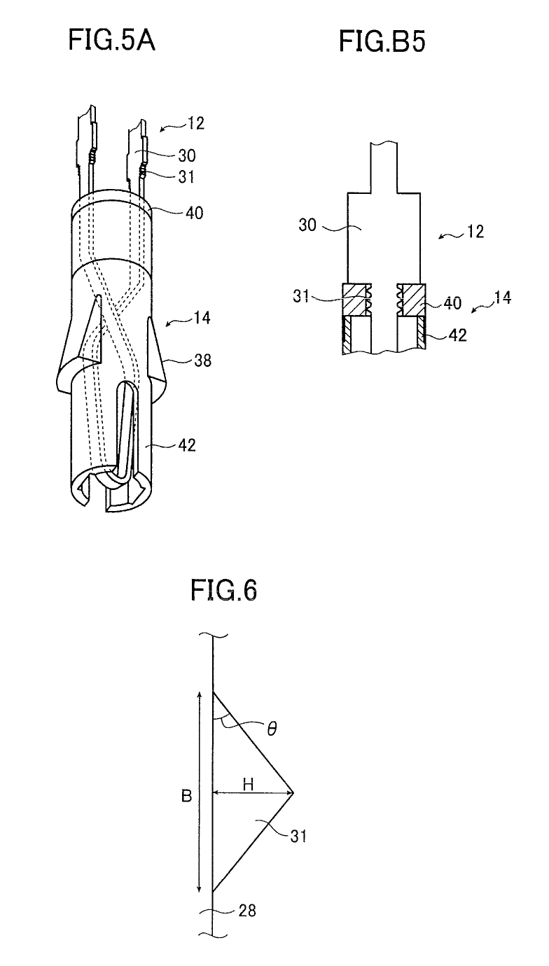

Next, a preferred embodiment of the micro projections 31 is described. FIGS. 5A and 5B are views illustrating clamping action of the clip 12 by the clamping portion 40 of the connection ring 14. FIG. 5A is a perspective view illustrating the state immediately before completion of clamping, and FIG. 5B is an enlarged cross-sectional view of a part of the projection portions 30 and the micro projections 31, illustrating the state upon completion of clamping.

As illustrated in FIG. 5A, the micro projections 31 are provided at the edges on both sides of each of the arm portions 28 of the clip 12 in the predetermined region on the proximal end side (rear end side) of each of the projection portions 30. The micro projections 31 are small protrusions protruding from the edge portions of each of the arm portions 28. The micro projections 31 increase a frictional coefficient with the inner wall of the clamping portion 40. In addition, as illustrated in FIG. 5B, the micro projections 31 generate the friction (galling) of metal portions with respect to the clamping portion 40 which has moved to the position of being brought into contact with the projection portions 30, and retain the clamping portion 40, that is, the connection ring 14 at the above-mentioned position, thereby preventing the connection ring 14 from moving in a direction of canceling the clamping of the clip 12 (on proximal end side).

FIG. 6 is an enlarged cross-sectional view of one of the micro projections 31.

The dimension of the micro projections 31 is set so as to exert a necessary frictional force by generating the friction (galling) with respect to the inner wall of the clamping portion 40. For example, in the clip 12 which is formed of SUS 631 and has the thickness of 0.14 mm and the width of 0.8 mm in the arm portions 28 in the vicinity of the projection portions 30, when the clamping portion 40 is formed of SUS 304, it is desirable that a protruding height H of the micro projections 31 be set to 0.05 to 0.1 mm, an angle θ with respect to a flat surface of the edge portion of each of the arm portions 28 be set to about 45°, and a longitudinal width B of the arm portions 28 be set to 0.1 to 0.2 mm.

Further, the range for providing the micro projections 31, the arrangement intervals thereof, and the number of the micro projections 31 are set so as to generate the frictional force or galling enough to retain the position of the clamping portion 40. For example, in the clip 12 having the above-mentioned dimension, when the clamping portion 40 has the inner diameter of 1.1 mm and the height of 1.4 mm, it is desirable that three micro projections 31 be provided in a range apart from each of the lower ends of the projection portion 30 by 0.3 to 0.5 mm in a moving direction of the connection ring 14 with respect to the clip 12. However, when the micro projections 31 are provided in a large range, a load for moving the connection ring 14 at the time of clamping of the clip 12 becomes high, whereby a manipulation force required to pull the manipulating wire 20 is high. Further, in the clip 12 after completion of clamping, when the micro projections 31 are provided in a large range, the connection ring 14 (clamping portion 40) is not easily shifted rearward, and hence it is difficult to remove the clip 12. Therefore, it is desirable that the micro projections 31 be provided in a range apart from a portion directly below the projection portion 30 by a length equal to or less than an axial length of the clamping portion 40 of the connection ring 14.

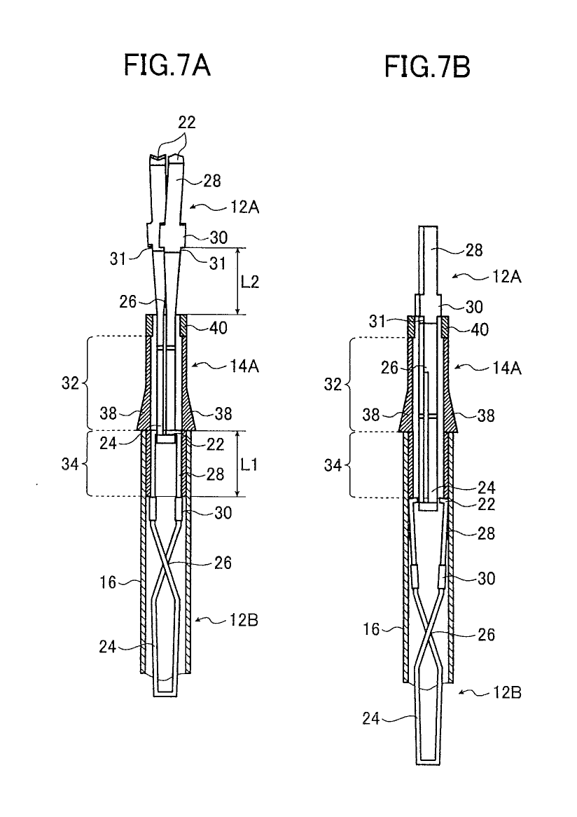

Next, structures of the clips 12 and the connection rings 14 and an operation of each component are described in detail while the connection ring 14A and the clips 12A, 12B retained by the same are given as an example. FIGS. 7A and 7B are partial cross-sectional views illustrating step wisely a state of the clips 12A, 12B and the connection ring 14A during clipping operation of the foremost clip 12A. FIG. 7A corresponds to an enlarged view of a forward end portion of FIG. 1A. Note that, in FIG. 7A, illustration of the connection ring 14B for retaining the clip 12B is omitted. Further, the following operation is the same in the subsequent other clips 12 and the connection rings 14.

In a state illustrated in FIG. 7A, the connection ring 14A is fitted onto the clip 12A and the clip 12B such that the clamping portion 40 thereof is situated at a predetermined initial position of the clip 12A. The initial position is in the vicinity of the crossing portion 26 of the clip 12A. The clamping portion 40 of the connection ring 14A does not clamp the clip 12A, and the arm portions 28, 28 of the clip 12A diverge at the maximum diverging amount.

The turned portion 24 of the clip 12A is accommodated in the first region 32 of the connection ring 14A, and the arm portions 28, 28 of the clip 12B are retained in the second region 34 of the connection ring 14A in a state in which the claw portions 22, 22 are closed while pinching the tail portion of the clip 12A. The forward end of the clip 12B is substantially matched with the upper end of the second region 34 of the connection ring 14A, and the upper ends of the projection portions 30 of the clip 12B are brought into contact with the lower end of the connection ring 14A. Therefore, a length L1 measured from the forward end of the clip 12B to the upper ends of the projection portions 30 is substantially the same as a region length of the second region 34 of the connection ring 14A.

In the state of FIG. 7A, a length L2 measured from the lower ends of the projection portions 30 of the clip 12A to the forward end of the connection ring 14A (clamping portion 40 thereof) is substantially equal to the length L1. The lengths L1 and L2 are equal to a relative moving amount of the connection ring 14A with respect to the clip 12A for clamping the clip 12A, and are substantially equal to the pulling amount of the manipulating wire 20 (see FIGS. 1A and 1B) for causing the clip 12A and the like to retreat with respect to the connection ring 14A and the like.

In the state of FIG. 7A, by pulling the manipulating wire 20 by the predetermined amount L2, the clip 12A is moved by the length L2 with respect to the connection ring 14A, and as illustrated in FIG. 7B, the lower ends of the projection portions 30 of the clip 12A are brought into contact with the forward end of the connection ring 14A. When the connection ring 14A is situated directly below the projection portions 30, the clip 12A exerts, in the claw portions 22, 22, a predetermined fit-engagement force, for example, the design upper limit value of the fit-engagement force of the clip 12A, and clamping of the clip 12A by the clamping portion 40 of the connection ring 14A is completed. At this time, the clamping portion 40 comes into contact with the micro projections 31 directly below the projection portions 30. The frictional force between both members is sufficiently high, and hence the clamping portion 40 is retained at the position directly below the projection portions 30.

Further, by pulling the manipulating wire 20 by the length L2, the clip 12B is also moved by the same amount as the clip 12A is moved. That is, the clip 12B is moved by the region length L1 of the second region 34 which is substantially equal to the length L2, and the forward end of the clip 12B is detached from the proximal end of the connection ring 14A. As a result, the engagement portion between the clip 12A and the clip 12B is detached from the second region 34 of the connection ring 14A.

In this way, in the initial state, the clamping portion 40 of the connection ring 14A is set at the fixed initial position of the preceding clip 12A, that is, at the position apart by the length L2 from the lower ends of the projection portions 30 of the clip 12A. The manipulating wire 20 is pulled by the fixed pulling amount (stroke) L2 every time, and the clamping portion 40 is moved to the lower ends of the projection portions 30 of the clip 12A, whereby clamping of the clip 12 can be completed.

As described above, in the clip 12, the clamping portion 40 of the connection ring 14 is moved toward the forward end of the clip 12, whereby the arm portions 28, 28 are gradually closed from the diverging state to hold an object to be treated between the claw portions 22, 22 provided on the forward end thereof. At this time, between the arm portions 28 and the inner wall of the clamping portion 40, a pressurizing force is exerted by an urging force (spring force) due to the bending rigidity (elasticity) of the arm portions 28.

Therefore, the clip 12 has the micro projections 31 directly below the projection portions 30, and hence, during clamping of the clip 12 by the movement of the clamping portion 40, rubbing of metal portions occurs between the micro projections 31 and the inner wall of the clamping portion 40 to thereby generate galling. Owing to the galling and the micro projections 31 themselves, the clamping portion 40 is locked at the position of being brought into contact with the projection portions 30, or at the position in the vicinity thereof, whereby it is possible to maintain the position of the clamping portion 40 upon completion of clamping. Therefore, in the claw portions 22, 22 of the clip 12, the clip 12 and the clamping portion 40 (connection ring 14) can easily and precisely exert the designed fit-engagement force, and can maintain the fit-engagement force.

Further, the clip 12 has the micro projections 31 locally in the range having the length equal to or less than that of the clamping portion 40. Accordingly, the manipulation force for clamping is not unnecessarily increased, and the clip 12 in which clamping has been completed once can be removed.

Note that, when a thickness of a portion of a living body (portion to be treated) to be held by the claw portions 22, 22 is large, bending deformation of the clip occurs at an initial stage in which the connection ring 14 passes through the arm portions 28, 28, and hence the arm portions 28 are pressed against the inner wall of the connection ring 14 more strongly. However, the arm portions 28, 28 of the clip 12 are formed such that, in a range of use thereof, plastic deformation does not occur, whereas elastic deformation occurs.

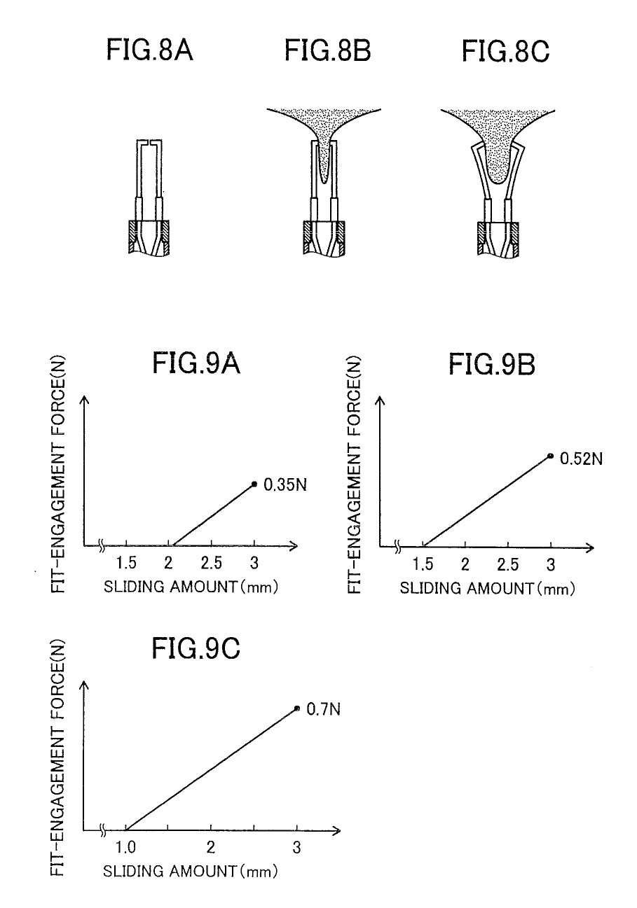

FIGS. 8A to 8C are schematic views illustrating states of the clip after completion of clamping by the clamping portion 40 of the connection ring 14. FIG. 8A illustrates a state in which nothing is pinched between the claw portions 22, 22, that is, a state in which the claw portions 22, 22 are held in contact with each other. FIG. 8B illustrates a state in which the clip 12 holds a small portion of the living body, and FIG. 8C illustrates a state in which the clip 12 holds a large portion of the living body. Further, FIGS. 9A to 9C are graphs showing a relation between a sliding amount of the connection ring 14 and a fit-engagement force generated between the claw portions 22, 22 of the clip 12 when a clamping state illustrated in each of FIGS. 8A to 8C is obtained. In FIGS. 9A to 9C, an axis of abscissa represents the sliding amount (mm) of the connection ring 14 and an axis of ordinate represents the fit-engagement force (N) of the clip 12.

In FIGS. 9A to 9C, premising that the initial position of the connection ring 14 in the initial state (see FIG. 7A) of the clipping device 10 is set to zero, the sliding amount of the connection ring 14 indicates the length by which the connection ring 14 moves toward the forward end of the clip 12 from the initial position, and the maximum value thereof is equal to the length L2 measured from the upper end of the connection ring 14 in the initial position to the lower ends of the projection portions 30. In examples illustrated in FIGS. 8A to 8C and shown in FIGS. 9A to 9C, L2=3 mm. Further, the fit-engagement force represents pressure generated between the claw portions 22, 22.

In the case where nothing is held as illustrated in FIG. 8A, as shown in FIG. 9A, the diverging amount of the claw portions 22, 22 becomes gradually small until the connection ring 14 is slid by 2.1 mm. However, the claw portions 22, 22 do not come into contact with each other yet, and hence the fit-engagement force is not generated therebetween. When the sliding amount becomes 2.1 mm, the fit-engagement force is generated between the claw portions 22, 22. After that, the fit-engagement force is increased proportionally, and at the point in time when the sliding amount reaches 3 mm, the fit-engagement force of 0.35 N is generated.

In the case where a portion to be subjected to clipping is small as illustrated in FIG. 8B, when the sliding amount of the connection ring 14 becomes 1.5 mm as shown in FIG. 9B, the fit-engagement force is generated between the claw portions 22, 22. After that, the fit-engagement force is increased proportionally, and at the point in time when the sliding amount reaches 3 mm, the fit-engagement force of 0.52 N is generated.

In the case where a portion to be subjected to clipping is large as illustrated in FIG. 8C, when the sliding amount of the connection ring 14 becomes 1.0 mm as shown in FIG. 9C, the fit-engagement force is generated between the claw portions 22, 22. After that, the fit-engagement force is increased proportionally, and at the point in time when the sliding amount reaches 3 mm, the fit-engagement force of 0.7 N is generated. In FIG. 8C, the arm portions 28 of the clip are curved inwardly so as to be opposed to each other. However, plastic deformation does not occur in the clip 12, and elastic deformation occurs in the range of use of the clip 12.

Next, there is described the case of removing the clip 12 in which clamping by the connection ring 14 has been completed once.

In the state in which clamping by the connection ring 14 has been completed, the arm portions 28, 28 of the clip 12 are pressed by the clamping portion 40 of the connection ring 14, and owing to the micro projections 31 of the arm portions 28 and the galling generated thereby, a large frictional force is exerted between the edge portions of the arm portions 28 and the inner wall of the clamping portion 40. Thus, if the clamping portion 40 of the connection ring 14 is merely pulled, it is impossible to easily cancel the clamping. However, as shown in FIGS. 9A to 9C, regardless of the size of the object to be treated, the clip 12 is used in its elastic deformation region, and hence the arm portions 28, 28 of the clip 12 can be elastically deformed again. Then, the vicinity of the projection portions 30 of the clip 12 situated at the forward end portion of the clamping portion 40 for clamping the clip 12 is pressed in a central direction of the clamping portion 40, that is, in a direction of closing the arm portions 28 of the clip 12, whereby the arm portions 28, 28 are slightly elastically deformed.

The arm portions 28, 28 of the clip 12 remain in a portion smaller than the inner diameter of the clamping portion 40. Thus, when the arm portions 28, 28 are caused to slightly enter the inside, the clamping portion 40 is shifted to the rear end side of the clip 12 to bring the clamping portion 40 and the arm portions 28, 28 out of contact with each other. As a result, retention by the galling is canceled to loosen the clamping of the clip 12 by the clamping portion 40. When the clamping portion 40 loosens even if only slightly, the arm portions 28, 28 diverge in the diverging direction, and hence the claw portions 22, 22 can be removed from the portion of the living body which is held by the claw portions 22, 22.

When pressurizing the arm portions 28 continuously, the clamping portion 40 moves greatly to the rear end side of the clip 12, and the claw portions 22, 22 of the clip 12 excessively diverge. In the most-diverging state, the clamping portion 40 is detached from the clip 12, and the arm portions 28 may exhibit a shape like a dogleg-shaped plate. It seems that there is a risk of damaging the living body due to the above-mentioned shape, and hence it is desirable that the pressurization of the arm portions 28 at one time be performed for a very short period of time so as to slightly move the clamping portion 40. When canceling the pressurization of the arm portions 28, the arm portions 28 diverge again by the own urging force to press the inner wall of the clamping portion 40. The clamping portion 40 remains at the position of being shifted slightly rearward, and the diverging amount of the clip 12 is maintained as it is.

In the case where performing the pressurization of the arm portions 28 once is insufficient for diverging of the clip 12, whereby the clip 12 cannot be removed from the holding portion, the pressurizing manipulation of the arm portions 28 may be performed intermittently and repeatedly more than once until the clip 12 is removed from the holding portion.

Note that, the clip 12 is a closed clip having the turned portion 24. At the time of clipping, the clip 12 is pulled strongly by the subsequent clip 12 or the dummy clip 18, and hence the rear end portion of the turned portion 24 is plastically deformed when clipping is completed, whereby the turned portion 24 does not widen greatly if the arm portions 28, 28 of the clip 12 are pressed. Therefore, a frictional force between the retaining portion 42 of the connection ring 14 and the turned portion 24 is not large enough to hinder the movement of the connection ring 14, and there is no problem of detaching the connection ring 14.

Further, the clamping portion 40 and the retaining portion 42 of the connection ring 14 are combined only through fitting to each other, and hence, after clamping of the clip 12, the retaining portion 42 may be detached from the clamping portion 40 to be discharged outside the living body in some cases. Note that, when detaching the connection ring 14, the retaining portion 42 may be first detached from the clamping portion 40, and then the clamping portion 40 may be detached from the clip 12.

In order to pressurize the vicinity of the projection portions 30 of the clip 12 in which clipping has been completed, there may be used a clipping device capable of an opening/closing manipulation. Further, the clip 12 and the connection ring 14 removed from the living tissue may be collected by a clipping device or the like.

### Second Embodiment

In the first embodiment, by provision of the micro projections 31 on the proximal end sides of the projection portions of the clip 12, the position of the clamping portion 40 of the connection ring 14 in the clip 12 is reliably maintained, and the clamping state of the clip is maintained. However, the present invention is not limited thereto, and, by provision of recesses on the rear end sides of the projection portions of the clip, the clamping state of the clip may be maintained.

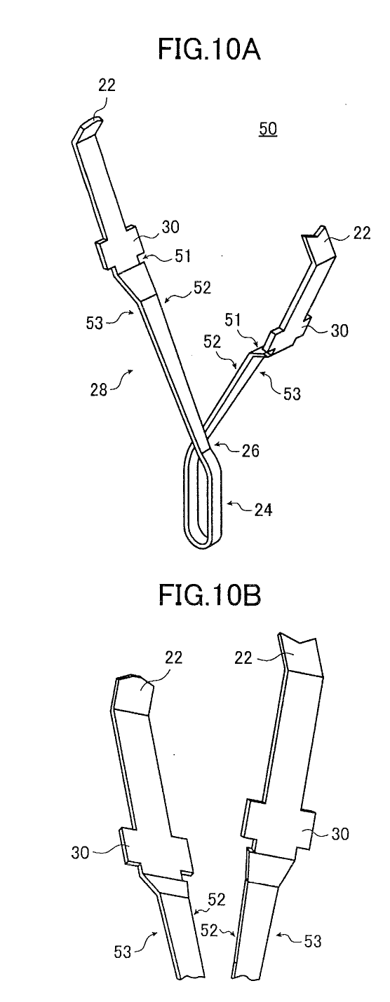

FIGS. 10A and 10B illustrate one of clips 50 as described above used in a second embodiment. In the clip 50 illustrated in FIGS. 10A and 10B, portions common to those of the clip 12 illustrated in FIG. 2 are denoted by the same reference symbols, and hence only different points are described.

The clip 50 has recesses 51 each formed at the rear end of each of the projection portions 30 at one edge of each of the arm portions 28. The rear end side of each of the recesses 51 has an inclined portion 52 inclined with respect to a straight portion 53 on the opposite side of each of the arm portions 28, and each of the arm portions 28 increases in width toward its forward end. Two arm portions 28, 28, recesses 51, 51, and inclined portions 52, 52 are arranged at point symmetry with respect to the center of the clip 50.

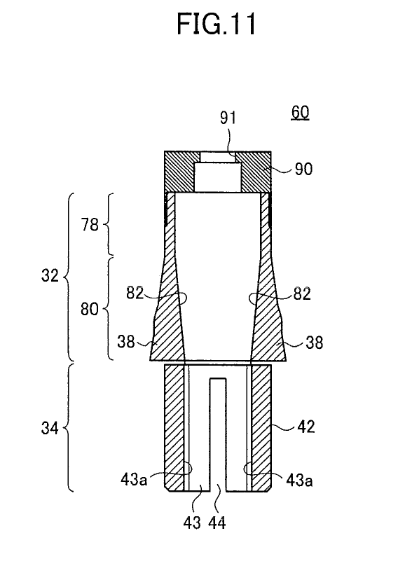

FIG. 11 is a cross-sectional view of a connection ring 60 used together with the clip 50 in the second embodiment. Here, in the connection ring 60 illustrated in FIG. 11, portions common to those of the connection ring 14 illustrated in FIG. 4B are denoted by the same reference symbols, and the connection ring 60 is different from the connection ring 14 in that a protrusion 91 smaller than the inner diameter of a clamping portion 90 is provided on the forward end side of the clamping portion 90 provided to the connection ring 60.

Here, shapes of the recesses 51 of the clip 50 and the protrusion 91 of the clamping portion 90 of the connection ring 60 are not particularly limited. As long as the protrusion 91 has a shape allowing fitting to the recesses 51, and fitting the protrusion 91 to the recesses 51 enables to maintain the clamping state of the clip, any shape may be adopted.

In the second embodiment, the same operation as that of the clip 12 and the connection ring 14 in the first embodiment is performed, with the exception that the operation by the micro projections 31 of the clip 12 and the operation by the recesses 51 of the clip 50 and the protrusion 91 of the connection ring 60 are different from each other.

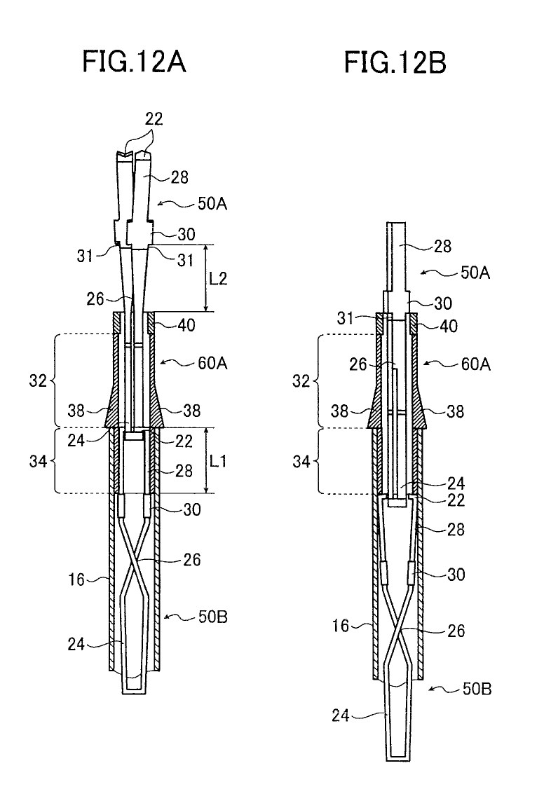

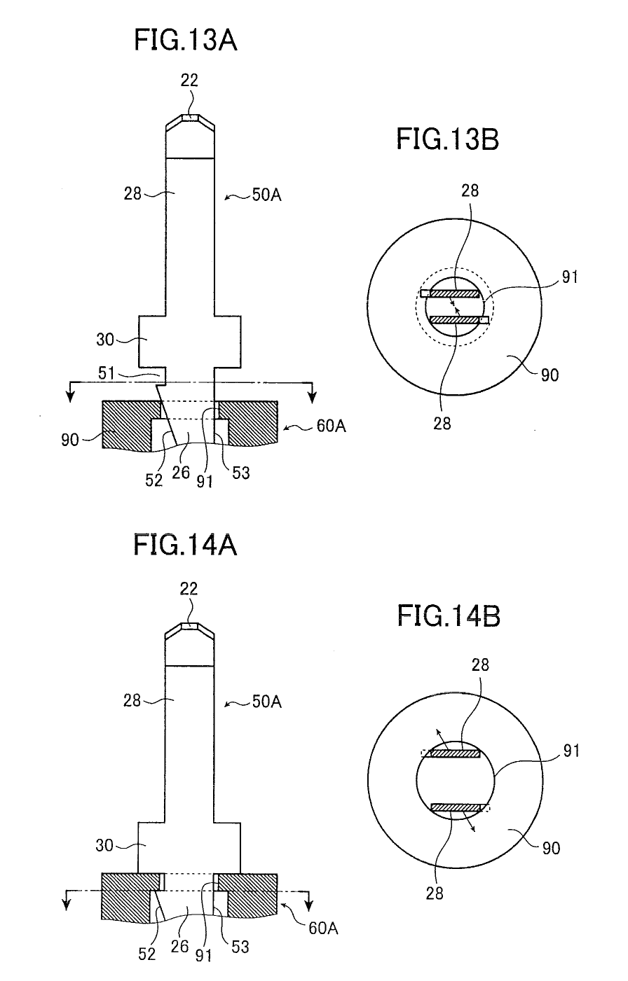

By pulling the manipulating wire 20 (see FIGS. 1A and 1B), as illustrated in FIGS. 12A and 12B, a clip 50A is pulled to the rear end side thereof, and a connection ring 60A is moved to the forward end side of the arm portions 28 of the clip 50A. When the connection ring 60A is moved, as illustrated in FIG. 13A, the forward end side of the protrusion 91 of the connection ring 60 is held in contact with the straight portions 53 and the inclined portions 52. Further, as illustrated in FIG. 13B, along with the movement of the connection ring, the arm portions 28 of the clip are pressed against the inner wall of the protrusion 91 of the connection ring 60A to move toward the center of the protrusion 91.

In addition, the connection ring 60A is moved to the forward end side of the arm portions 28 of the clip 50A, as illustrated in FIG. 14A, the connection ring 60A is brought into contact with the projection portions 30 of the clip 50A, and then the protrusion 91 of the connection ring 60A is fitted on the rear end sides of the recesses 51 of the clip 50A. At this time, the arm portions 28 of the clip 50A are moved outside as illustrated in FIG. 14B, and the positions of the arm portions 28 are fixed to performed clamping with a predetermined clamping force, whereby the clamping force is maintained.

When pulling the manipulating wire 20, the inner wall of the protrusion 91 of the connection ring 60A receives the pressure applied to the outer side of the arm portions 28 of the clip 50. However, the straight portions 53 and the inclined portions 52 of the arm portions 28 have no projection and no recess, and hence a frictional force received thereby is small. Further, while the turned portion 24 is moved upward by the inclined portions 52, 52 of the two arm portions 28, 28 of the clip 50, the edges of the arm portions 28 are inclined, whereby a contact force in the turned portion 24 can be reduced. Therefore, a force necessary for pulling the manipulating wire 20 can be reduced.

In the second embodiment, there is adopted the structure in which the protrusion 91 of the connection ring 60A is fitted to the recesses 51 of the clip 50, and hence the total length of the clips can be reduced when compared with the structure in which the entire clamping portions 90 of the connection rings 60 having no protrusion are fitted to each other.

The clip 50 is manufactured by, for example, punching a strap-shaped plate. In this case, portions corresponding to the projection portions 30 and the recesses 51 are provided in advance, and the punched plate is bent to manufacture the clip. It is desirable that the rear end side height of the recesses 51 be approximately 0.1 mm. The recesses 51 do not exhibit a micro shape, and hence additional working is unnecessary and manufacturing property of the clip 50 is high.

Note that, in the second embodiment, there is adopted the structure in which the protrusion 91 of the connection ring 60A is fitted to the recesses 51 of the clip 50. However, the present invention is not limited thereto, and there may be adopted the structure in which the entire clamping portions 90 of the connection rings 60 having no protrusion are fitted to the recesses 51 of the clips 50. Further, the inclined portions 52 of the clip 50 may be parallel to the straight portions 53. In addition, the recesses 51 of the clip 50 may be formed not only at one edge of each of the arm portions 28 but also at both edges thereof.

While the successive clipping device is described above, the present invention can be also applied to the single clipping device. In the case of the single type, for example, there may be assumed a clipping device in which only the rearmost clip 12D and connection ring 14D of the above-mentioned clipping device 10 are loaded into the sheath 16.

Note that, in the first and second embodiments, the clips 12, 50 are connected together, with their orientations alternately differing by 90 degrees. However, the present invention is not limited thereto, and the shape of the interior of the connection ring may be selected in accordance with the shape of the engagement portion. For example, a clip having a shape twisted by 90 degrees at a portion between the claw portions 22, 22 and the turned portion 24 is used, and the successive clips may be connected together, with their orientations being the same. Further, the present invention is desirable in that, by using the closed clip having the turned portion, it is possible to impart the spring force (urging force) to diverge the arm portions by pressurizing the turned portion. It goes without saying that the present invention may be applied to a clipping device using an open clip (U-shaped clip) having no turned portion.

The successive clipping device of the present invention described in detail above should not be construed restrictively. It goes without saying that various improvements and variations are possible without departing from the gist of the present invention. The successive clipping device of the present invention is applicable not only to a soft endoscope but also to a hard endoscope.

What is claimed is:

1. A clipping device comprising:
a clip having a pair of arm portions diverging elastically; and
a tubular clamping member fitted onto the pair of arm portions of the clip, for releasing the clip when being situated on a proximal end side of the pair of arm portions so as to cause the pair of arm portions to diverge, and for clamping the clip when being situated on a forward end side of the pair of arm portions so as to cause the pair of arm portions to close,
each of the pair of arm portions including a projection portion which is formed in a vicinity of a forward end portion thereof to have a width inhibiting passage of the clamping member, and an engagement portion which is formed in a region adjacent to a proximal end side of the projection portion to be engaged with the clamping member,
the clamping member being engaged with the engagement portion of each of the pair of arm portions to maintain clamping of the clip so that the pair of arm portions holds an object to be treated.

2. The clipping device according to claim 1, wherein the engagement portion comprises micro projections formed on both edge portions of each of the pair of arm portions.

3. The clipping device according to claim 1, wherein the engagement portion has a length equal to or less than a length of the clamping member.

4. The clipping device according to claim 1, wherein the engagement portion comprises a recess formed on one of both the edge portions of each of the pair of arm portions.

5. The clipping device according to claim 4, wherein the clamping member comprises an annular protrusion protruding in an inner circumferential direction, the annular protrusion being engaged with the recess of each of the pair of arm portions to maintain the clamping of the clip.

6. The clipping device according to claim 4, wherein each of the pair of arm portions comprises a tapered portion which is formed in a region adjacent to a proximal end side of the recess and which decreases in width toward the proximal end side.

7. The clipping device according to claim 1, wherein each of the pair of arm portions comprises a claw portion formed on the forward end portion thereof, for holding the object to be treated.

8. The clipping device according to claim 1, further comprising:
a sheath having a proximal end portion and a forward end portion, in which the clip and the clamping member fitted onto the clip are loaded into the forward end portion thereof; and
a manipulating wire connected to the clip, for pulling the clip with respect to the sheath.

9. A clipping device comprising:
a sheath having a proximal end portion and a forward end portion;
a plurality of clips which are loaded into the forward end portion of the sheath while being engaged with other clips connected together in front and back directions, and each of which comprises a pair of arm portions diverging elastically;
a plurality of tubular clamping members corresponding to the plurality of clips and fitted into the sheath so as to be capable of advancing and retreating, each being fitted onto the pair of arm portions of the corresponding clip, each releasing the corresponding clip when being situated on a proximal end side of the pair of arm portions so as to cause the pair of arm portions to diverge, and each clamping the corresponding clip when being situated on a forward end side of the pair of arm portions so as to cause the pair of arm portions to close;
a plurality of retaining members corresponding to the plurality of clamping members and connected to the corresponding clamping members, and each covering an engagement portion of the clip, onto which the corresponding clamping member is fitted, so as to maintain the plurality of clips in a connected state; and
a manipulating wire connected to a rearmost one of the plurality of clips, for pulling a clip string constituted by the plurality of clips,
each of the pair of arm portions of each clip including a projection portion which is formed in a vicinity of a forward end portion thereof to have a width inhibiting passage of the clamping member, and an engagement portion which is formed in a region adjacent to a proximal end side of the projection portion to be engaged with the clamping member,
each of the clamping members being engaged with the engagement portion of each of the pair of arm portions of the corresponding clip to maintain clamping of the clip.

10. The clipping device according to claim 9, wherein:
the pair of arm portions of each of the clips crosses each other and each comprises claw portions formed on the forward end portion thereof,
each of the clips including a turned portion connecting the pair of arm portions crossing each other,
a preceding clip and a subsequent clip connected together in the front and back directions being engaged with each other by closing the pair of arm portions of the subsequent clip so that the claw portions of the pair of arm portions of the subsequent clip pinch the turned portion of the preceding clip.
